(19) United States
(12) Patent Application Publication
(10) Pub. No.: US 2010/0204552 A1
(43) Pub. Date: Aug. 12, 2010

(54) APPARATUS FOR MONITORING BIOLOGICAL INFORMATION

(75) Inventors: Norihito Yamamoto, Shiga (JP); Ryo Fukui, Kyoto (JP); Midori Masuno, Kyoto (JP); Hiromichi Karo, Kyoto (JP); Masahiro Umeda, Kyoto (JP); Noboru Shinomiya, Osaka (JP)

Correspondence Address: RABIN & Berdo, PC, 1101 14TH STREET, NW, SUITE 500, WASHINGTON, DC, 20005, US

(73) Assignee: Omron Healthcare Co., Ltd., Kyoto-shi (JP)

(21) Appl. No.: 12/671,516

(22) PCT Filed: Jun. 2, 2008
(86) PCT No.: PCT/JP2008/060154

§ 371 Date: Jan. 31, 2010

(30) Foreign Application Priority Data
Jul. 30, 2007 (JP) 2007-197803

(51) Int. Cl. A61B 5/00

(52) U.S. Cl. 600/301

(57) ABSTRACT

An apparatus for monitoring biological information is provided. The apparatus includes a detection part configured to detect a signal indicative of the biological information of a subject and a judging part configured to judge the biological information to identify an attribute of the biological information. The apparatus further includes a storage part configured to store the attribute of the biological information together with a time that the attribute is stored and a producing part configured to produce a signal to display the attribute in a chart defined by a first axis representing a cyclic time series and a second axis representing a time series within a cycle of the cyclic time series.

### BACKGROUND

The invention is related to an apparatus for monitoring biological information, a method of displaying biological information, and a data storage medium recording a computer program for displaying biological information.

Electrocardiographic monitoring and storing devices (hereinafter referred to as an electrocardiographs), blood pressure monitoring devices (hereinafter referred to as sphygmomanometers), and sphygmographic monitoring devices (hereinafter referred to as sphygmographs) are typical examples of apparatus for monitoring patient biological information a. Types of examination for monitoring an electrocardiogram using an electrocardiograph include 12 lead electrocardiography in a hospital and Holter electrocardiography for 24 hour recording. However, in recent years, with regard to the less common disorder of arrhythmia, which is hard to discover even by these types of examination, the usefulness of an event electrocardiogram is becoming more widely recognized.

Above all else, a relapse in an arrhythmia, etc. after a cardiac operation has previously been diagnosed only from the patient's subjective symptoms. However, such a relapse can actually occur without subjective symptoms. Further, it is becoming clear that a relapse can also occur, not just immediately after the operation, but a few days afterwards. Therefore, the usefulness of an event cardiogram for discovering these relapses is becoming recognized. The same relapses as those seen after the operation are considered to take place during a follow-up treatment with medication.

However, in order to perform these follow-ups using a portable electrocardiograph such as an event electrocardiograph, the patient is supposed to always carry the portable electrocardiograph during a period of two weeks to one month and operate it to record the electrocardiogram by himself or herself when the patient recognizes the subjective symptoms or at timings advised by a medical doctor (for example, first thing in the morning, before bedtime, etc.). The electrocardiogram as recorded above needs some tens of seconds or a few minutes for one recording, however the total number of recordings during the period when the patient carries the portable electrocardiograph reaches as many as some hundreds of times.

Much the same is true for other apparatus. For example, sphygmomanometers as well as sphygmographs are getting downsized to be used for home medical care in recent years. These apparatus are used in order to record blood pressures or pulse waves first thing in the morning or before bedtime, etc. at timings advised by a medical doctor or when a patient recognizes predetermined subjective symptoms. Accordingly, the total number of recordings during the period when the patient carries them reaches as many as some hundreds of times as well.

The patient brings the data monitored and recorded as above to a hospital to have a checkup by a medical doctor. If the doctor review all the data during an examination, it is extremely difficult for him or her to pick out data useful for a clinical examination, such as important symptoms, out of the huge volumes of data.

### BRIEF SUMMARY

According to one aspect of the invention, there is provided an apparatus for monitoring biological information, including a detection part configured to detect a signal indicative of the biological information of a subject, a judging part configured to judge the biological information to identify an attribute of the biological information, a storage part configured to store the attribute of the biological information together with a time that the attribute is stored, and a producing part configured to produce a signal to display the attribute in a chart defined by a first axis representing a cyclic time series and a second axis representing a time series within a cycle of the cyclic time series.

According to another aspect of the invention, there is provided a method of displaying biological information obtained by an apparatus for monitoring biological information, including the steps of obtaining an attribute of the biological information from a storage part together with a time that the attribute is stored and producing a signal to display the attribute in a chart defined by a first axis representing a cyclic time series and a second axis representing a time series within a cycle of the cyclic time series.

According to still another aspect of the invention, there is provided a data storage medium recording a computer program for displaying biological information obtained by an apparatus for monitoring biological information, the computer program when executed by a computer, causing the computer to perform a method including a step of obtaining an attribute of the biological information together with a time that the attribute is stored, from a storage part of the computer, and a step of producing a signal to display the attribute in a chart defined by a first axis representing a cyclic time series and a second axis representing a time series within a cycle of the cyclic time series.

### BRIEF DESCRIPTION OF THE DRAWINGS

FIG. 1 is a schematic perspective view of a portable electrocardiograph according to one embodiment of the invention.

FIG. 2 is a schematic perspective view of a portable electrocardiograph according to one embodiment of the invention.

FIG. 3 is a perspective view illustrating a monitoring posture of a subject.

FIG. 4 is a view of the monitoring posture of FIG. 3 viewed from above.

FIG. 5 is a functional block diagram illustrating the functional structure of a portable electrocardiograph according to one embodiment of the invention.

FIG. 6 is a flowchart illustrating a monitoring process of the electrocardiographic waveform in a portable electrocardiograph according to one embodiment of the invention.

FIG. 7 is a display example during monitoring using a portable electrocardiograph according to one embodiment of the invention.

FIG. 8A is a view illustrating a judging process of a type of an electrocardiographic waveform in a portable electrocardiograph according to one embodiment of the invention.

FIG. 8B is a view illustrating a judging process of a type of an electrocardiographic waveform in a portable electrocardiograph according to one embodiment of the invention.

FIG. 9 is a view illustrating a specific example of the data structure of monitored results stored in a nonvolatile memory.

FIG. 10 is a functional block diagram illustrating a functional structure for displaying the monitored results stored in FIG. 9 using a CPU functioning as a display processing part.

FIG. 11 is a flowchart illustrating a displaying process of monitored results in a portable electrocardiograph according to one embodiment of the invention.

FIG. 12 is a view of a display example displayed in a display part.

FIG. 13 is a schematic framework of an electrocardiographic waveform display system SYS, a typical example of a biological information display system according to a second embodiment of the invention.

FIG. 14 is a view of schematic configuration illustrating a computer hardware constituting a display unit according to one embodiment of the invention.

FIG. 15 is a functional block diagram illustrating a functional structure for a display part according to one embodiment of the invention.

FIG. 16 is a view illustrating a display example of monitored results displayed in a monitor.

### DETAILED DESCRIPTION

Hereinafter, embodiments of the invention are described with reference to the drawings. The same symbols are applied to the same parts and components in the following description. These names and functions are the same as well.

### First Embodiment

### Structure of Outer Appearance of Portable Electrocardiograph

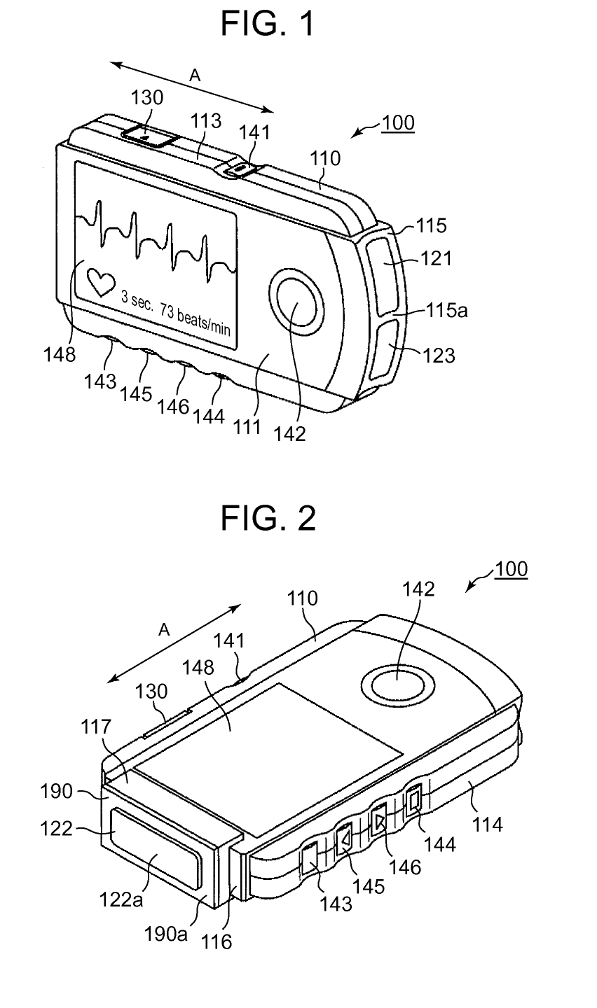

FIGS. 1 and 2 are schematic perspective views of a representative example of a biological monitoring apparatus and a portable electrocardiograph 100 as a typical example of a portable monitoring apparatus according to a first embodiment. The portable electrocardiograph 100 is principally used for event electrocardiographic testing to monitor an electrocardiographic waveform as a representative example of the biological information through an operation by a subject himself or herself.

The portable electrocardiograph 100 according to the present embodiment is reduced in size and weight to the extent that it can be held in one hand and can be best used as a portable apparatus. The portable electrocardiograph 100 has an apparatus body 110 formed in a flat and elongated substantially rectangular shape with its outer surface having a display part, operating part and monitoring electrode, etc.

A monitor button 142 is provided on one end of a front surface 111 in a longitudinal direction (in a direction of arrow A) of the portable electrocardiograph 100 to instruct a start of monitoring. A display part 148 is provided on the other end. The display part 148 is made up of, for example a LCD which displays monitor results and data entry screens for entering status values of a subject.

The monitor results are displayed as an electrocardiographic waveform or numerical data as shown in FIG. 1.

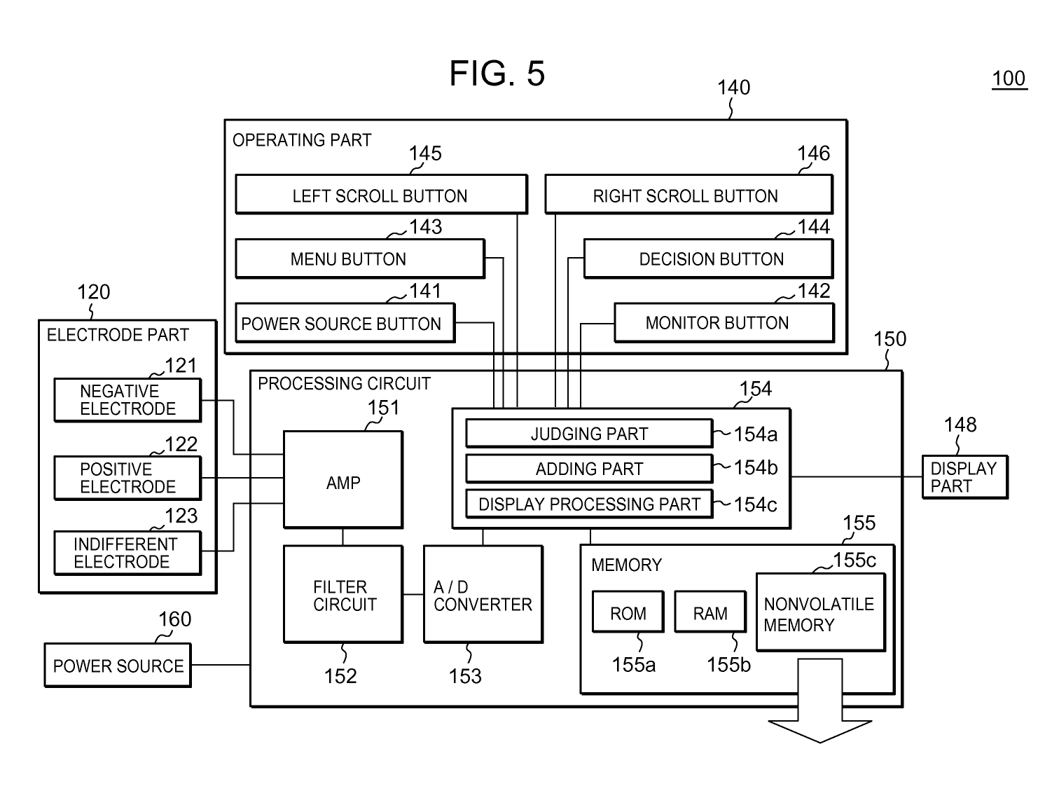

A power source button 141 is disposed in a predetermined position of a top face 113 of the apparatus body 110. The power source button 141 is an operating button for operating ON/OFF of the portable electrocardiograph 100. Further, a nonvolatile memory 155c (FIG. 5) included in the apparatus body 110 is detachable and a slot for attaching the nonvolatile memory 155c is provided on the apparatus body 110. For example, SD (Secure Digital) card is used as a nonvolatile memory 155c. An access cover 130 is provided as a lid on a predetermined position on the top face 113 of the apparatus body 110. The access cover 130 is provided to cover the slot when it is closed and attached to the apparatus body 110 such that it can be opened and closed freely.

There are located various operating buttons at a predetermined position on the bottom face 114 of the apparatus body 110. Menu button 143, decision button 144, left scroll button 145, and right scroll button 146 are disposed on the portable electrocardiograph 100 as shown in the drawings. The menu button 143 is an operating button for displaying various menu of the portable electrocardiograph 100. The decision button 144 is an operating button for performing menu or other respective operations. The left and right scroll buttons are operating buttons for scrolling and displaying charts as monitored results and guide information, etc. in the display part 148.

Negative electrode 121, being one of a pair of monitor electrodes, and indifferent electrode 123, for introducing a reference electrical potential with respect to a potential variation of a body, are located on a right lateral face 115 located at one end of the apparatus body 110 in a longitudinal direction. The right lateral face 115 is configured to have a contoured surface to fit with an index finger of the right hand of a subject when the subject takes a posture of monitoring as described later. The right lateral face 115 has a concave portion 115a elongated vertically. The concave portion 115a is configured to accept the index finger of the right hand of the subject.

The above-mentioned negative electrode 121 and indifferent electrode 123 are made of electrically conductive materials. Further, the negative electrode 121 and indifferent electrode 123 are configured such that their surfaces are exposed on the outer surface of the apparatus body 110 in the concave portion 115a provided on the right lateral face 115. The negative electrode 121 is positioned near the top face 113 on the right lateral face 115. The indifferent electrode 123 is positioned near the bottom face 114 on the right lateral face 115.

Positive electrode 122, being the other electrode of the pair of monitor electrodes, is located on the left lateral face 116 located at the other end of the apparatus body 110 in a longitudinal direction.

### (Monitoring Using Portable Electrocardiograph)

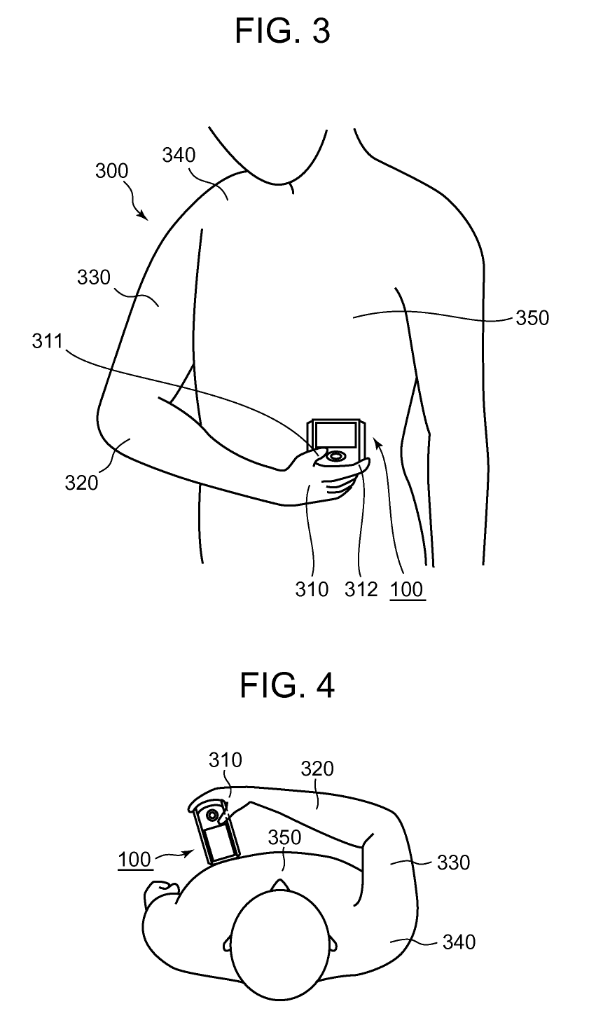

FIG. 3 is a perspective view illustrating a monitoring posture of a subject using a portable electrocardiograph 100 according to the present embodiment. FIG. 4 is a view of the monitoring posture of FIG. 3 viewed from above.

With reference to FIGS. 3 and 4, the subject 300 holds one end of the apparatus body 110 of the portable electrocardiograph 100 with the right hand while the subject 300 contacts the positive electrode 122 (FIG. 2) on the left lateral face 116 located at the other end of the apparatus body 110 directly to the skin on the line of the fifth intercostal front armpit positioned in a lower left part of breast 350. Then, the subject presses monitor button 142 (FIGS. 1 and 2) located on front face 111 of the apparatus body 110 with a thumb 311 of the right hand 310. Then, the subject monitors an electrocardiographic waveform keeping this state for some tens of seconds.

The above-mentioned monitoring posture maintains a state where the negative and indifferent electrodes located on the right lateral face 115 of the apparatus body 110 of the portable electrocardiograph 100 contact the index finger 312 of the right hand 310 of the subject 300, while the positive electrode 122 located on the left lateral face 116 of the apparatus body 110 contacts the breast 350 of the subject 300. In this situation, a monitor circuit is constituted in a body of the subject in series from the right hand 310 contacting to the negative electrode 121, the lower arm 320 not contacting to the breast 350, the upper arm 330 not contacting to the breast 350, the right shoulder 340, and the breast 350 to which the positive electrode 122 is attached.

In this way, the negative electrode 121, positive electrode 122 and indifferent electrode 123 detect a biological signal as an electrical signal from a part of the body of the subject 300.

### (Functional Structure of Portable Electrocardiograph)

FIG. 5 is a functional block diagram illustrating the functional structure of the portable electrocardiograph 100 according to one embodiment of the invention.

With reference to FIG. 5, the portable electrocardiograph 100 includes an electrode part 120 having the negative electrode 121, the positive electrode 122 and the indifferent electrode 123, and an operating part 140 having the power source button 141, the monitor button 142, the menu button 143, decision button 144, the left scroll button 145, and the right scroll button 146, the display part 148, power source 160, and processing circuit 150.

The processing circuit 150 includes an amplifier circuit 151 for amplifying a biological signal (electric signal) detected by the electrode part 120, a filter circuit 152 for eliminating noise content, an A/D converter 153 for converting analogue signals to digital signals, a CPU 154, and a memory 155. The memory 155 includes ROM 155a, RAM 155b, and nonvolatile memory 155c. The nonvolatile memory 155c is configured detachably with respect to the slot (not shown) as described above.

A biological signal (electric signal) detected by the electrode part 120 has its noise content eliminated by the filter circuit 152 after being amplified by the amplifier circuit 151. Then, it is converted to electrocardiographic waveform data (digital data) by the A/D converter 153. The CPU 154 stores the electrocardiographic waveform data converted by the A/D converter 153 into the nonvolatile memory 155c. The CPU 154 receives instruction signals from various operating buttons included in the operating part 140 and executes programs according to the instructions, while it controls display for the display part 148.

The CPU 154 includes a judging part 154a, an adding part 154b and a display processing part 154c as its functions. Typically, these functions are realized by the CPU 154 that retrieves programs initially stored in the ROM 155a to the RAM 155b and executes them.

The judging part 154a analyzes an electrocardiographic waveform outputted from the A/D converter 153 to identify a type of the electrocardiographic waveform. The judging part 154a transmits the identified results to the adding part 154b. The adding part 154b adds the attribute information including the type data provided from the judging part 154a and recording time provided from a timer (not shown) of the CPU 154 to the monitored results 280 provided from electrocardiographic waveform, then stores them in the nonvolatile memory 155c. The data structure of the monitored results is described later.

The display processing part 154c controls retrieval from the nonvolatile memory 155c of the electrocardiographic waveform to which the attribute data is added, corresponding to the operating signal provided from the operating part 140, and then displays the electrocardiographic waveform in the display part 148. The structure of the CPU 154 for controlling the display is described later as the display processing part 154c.

Further, the CPU 154 includes a timer (not shown) therein, obtains the present time. The time setting of the timer is made by a control signal from the CPU 154.

### (Monitoring Process)

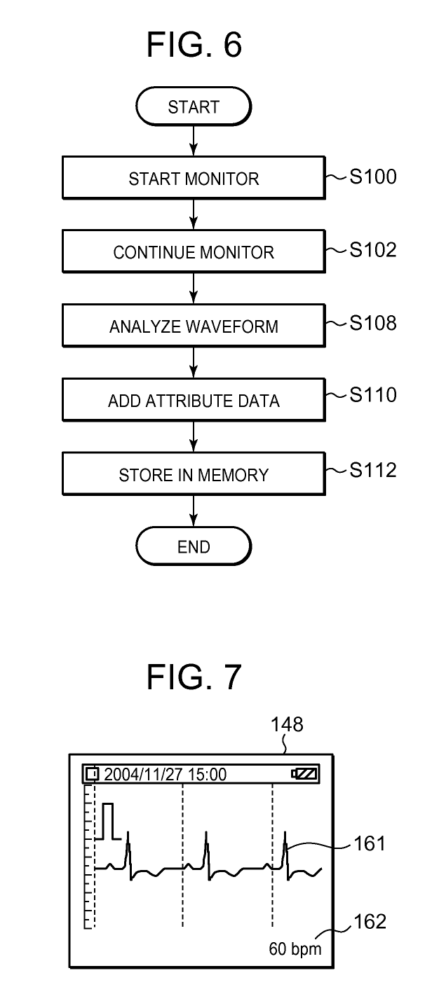

FIG. 6 is a flowchart illustrating a monitoring process of the electrocardiographic waveform in a portable electrocardiograph 100 according to one embodiment of the invention.

S100: With reference to FIG. 6, the CPU 154 starts to monitor the electrocardiographic waveform of a subject upon pressing the monitor button 142 (FIGS. 1 and 2). More specifically, the amplifier circuit 151 amplifies the electric signals detected by the electrode part 120, and the filter circuit 152 eliminates noises for the amplified electric signals. The A/D converter 153 converts the electric signals (analog signals) from which the noises are eliminated to digital signals. The CPU 154 receives the digital signals converted by the A/D converter 153 and displays them in the display part 148 in real time, while storing them in the RAM 155b temporarily.

S102: The above-mentioned processes are continuously executed for a predetermined time using a timer (not shown).

FIG. 7 is a display example of the display part 148 during monitoring using the portable electrocardiograph 100 according to one embodiment of the invention.

With reference to FIG. 7, the display part 148 displays the monitored electrocardiographic waveform 161 for a predetermined time period, while updating the display at any time to show a new electrocardiographic waveform by right or left scrolling according to the progress of monitoring. Further, a cardiac rate 162 is displayed on the same screen as the electrocardiographic waveform 161. The displayed rate of 60 bpm stands for 60 beats per minute. The item displayed during monitoring is not limited to the cardiac rate. Other items may be displayed appropriately.

S108: With reference to FIG. 6 again, the CPU 154 functioning as the judging part 154a executes analysis for the electrocardiographic waveform stored in the RAM 155b to identify a type of the electrocardiographic waveform. More specifically, the CPU 154 functioning as the judging part 154a identifies the type of the electrocardiographic waveform by extracting characteristic time changes appearing in the electrocardiographic waveform stored in the RAM 155b.

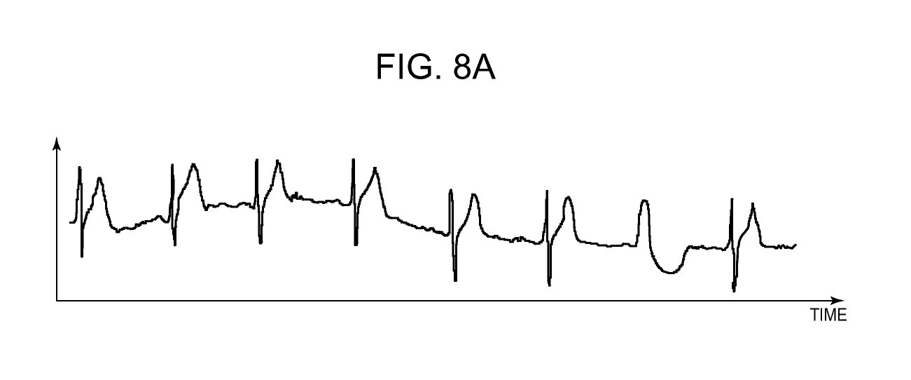

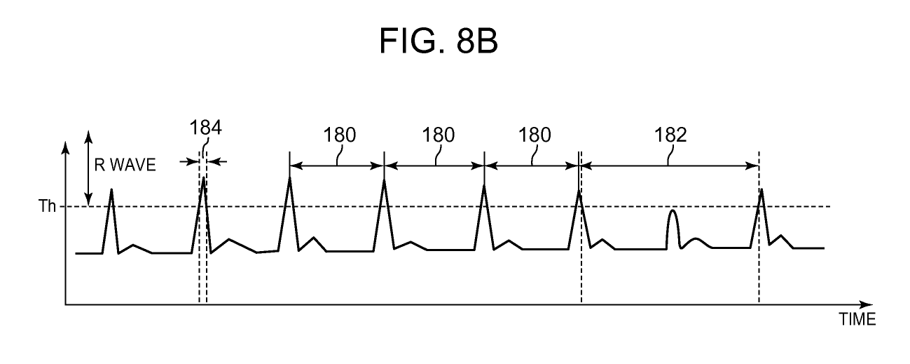

FIGS. 8A and 8B are views illustrating a judging process of a type of the electrocardiographic waveform in a portable electrocardiograph according to one embodiment of the invention. FIG. 8A shows a waveform of a biological signal (electric signal) detected by the electrode part 120 and FIG. 8B shows a waveform after the biological signal shown in FIG. 8A is filtered.

With reference to FIGS. 8A and 8B, the CPU 154 functioning as the judging part 154a extracts low-frequency components of the biological signals detected by the electrode part 120 as shown in FIG. 8A by adopting a filtering process corresponding to a kind of a Low pass filter (LPF) to generate a time waveform as shown in FIG. 8B. Further, the CPU 154 extracts components exceeding the predetermined threshold Th as R-wave components for the time waveform shown in FIG. 8B. In this way, the CPU 154 functioning as the judging part 154a extracts the characteristic time changes appearing in the biological signals detected by the electrode part 120.

Further, the CPU 154 processes to detect, for example an arrhythmia based on the R-wave components. The types of arrhythmia, for example are bradycardia (slow pulse), tachycardia (fast pulse), supraventricular premature beat and premature ventricular contraction (PVC). The CPU 154 is supposed to identify 5 types including bradycardia (slow pulse), tachycardia (fast pulse), supraventricular premature beat and premature ventricular contraction (PVC), and normal sinus rhythm (NSR) representing a normal electrocardiographic waveform. Identified items are not limited to these types. Other items may be added appropriately.

The above-mentioned five types are briefly described below. Generally, pulse rates of 50 or less per minute are termed bradycardia, and pulse rates of 100 or more per minute are termed tachycardia. Bradycardia and the tachycardia are judged based on the wave intervals 180 shown in FIG. 8B. The presence of irregular R-wave intervals is termed supraventricular premature beat, specifically a disrupted R-wave rhythm or missing one beat. The supraventricular premature beat is judged based on the wave intervals 180 of R-waves shown in FIG. 8B. Ventricular ectopy is termed a premature ventricular contraction (PVC) or VPC (Ventricular Premature Contraction). The premature ventricular contraction (PVC) is judged based on the width (QRS width) 184 of a peak triangle of R-wave as shown in FIG. 8B. The process of identifying the arrhythmia may be executed by using template matching. Specifically, preliminarily storing the templates representing the waveforms as references for identifying the type of the arrhythmia, the monitored electrocardiographic waveforms may be judged by matching the stored templates.

The CPU 154 functioning as judging part 154a identifies the types of the monitored electrocardiographic waveforms based on the judging process described above.

S110: With reference to FIG. 6 again, receiving the type of the electrocardiographic waveform in step S108, the CPU 154 functioning as adding part 154b adds the type data as the attribute data to the electrocardiographic waveform data stored in the RAM 155b.

S112: And then, the CPU 154 stores the data in the nonvolatile memory 155c.

More specifically, the attribute data is added to the electrocardiographic waveform data as header information. Then, the monitoring process of the electrocardiographic waveform in the portable electrocardiograph 100 is terminated.

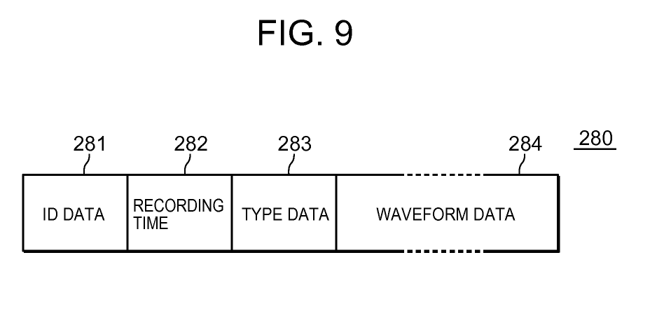

FIG. 9 is a view illustrating a specific example of the data structure of monitored results 280 stored in the nonvolatile memory 155c.

With reference to FIG. 9, the respective monitored results 280 stored in the nonvolatile memory 155c includes four fields 281 to 284 for “ID data”, “recording time”, “type data”, and “waveform data” as one example. The respective fields are described below. The ID data field 281 stores ID numbers or the like, identifying respective electrocardiographic waveforms. The recording time field 282 stores such data as monitor start time or monitor period for the respective electrocardiographic waveforms. The type data field 283 stores the type data identified by judging process in the judging part 154a. Further if the portable electrocardiograph 100 is configured to monitor electrocardiographic waveform for an identified subject by operating a button provided in the operating part 140 for designating the subject, the CPU 154 functioning as adding part 154b may add the ID number for identifying the subject to the monitored results. In this case, the ID number for identifying the subject is also stored in the above-mentioned ID data field 281.

In the example shown in FIG. 9, the electrocardiographic waveform is included as monitored results. However, the electrocardiographic waveform is not necessarily included if at least “recording time” and “type data” are included.

### (Function Structure for Display Process)

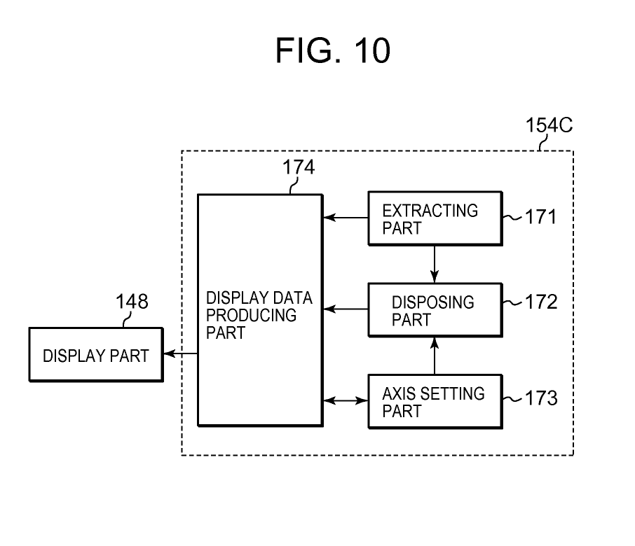

FIG. 10 is a functional block diagram illustrating a functional structure for displaying the monitored results stored in FIG. 9 using the CPU 154 functioning as the display processing part 154c.

With reference to FIG. 10, the CPU 154 functioning as the display processing part 154c includes extracting part 171, disposing part 172, axis setting part 173, and display data generating part 174 as its functions.

The extracting part 171 displays in the display part 148 an entry screen (not shown) for entering the period during which the monitored results are displayed. The display data generating part 174 preliminarily stores the display data for displaying the entry screen. The extracting part 171 sends to the display data producing part 174 the control signal to display the entry screen. The extracting part 171 receives the operating signal from the operating part that is entered in accordance with the entry screen and defines the period during which the monitored results are displayed in accordance with the operating signal. And, then the operating part 140 extracts from the nonvolatile memory 155c the monitored results within the defined period. More specifically, the extracting part 171 extracts from the monitored results 280 the corresponding monitored results in which the recording time stored in the field 282 (FIG. 9) is within the defined period. At that time, the extracting part 171 may extract the monitored results in which the waveform data stored in the field 284 meets a predetermined extracting condition within the defined period. The upper or lower limits of vibration amplitude specified for removing the effect of body motion etc, are applied as the extracting conditions. The extracting conditions are preliminarily stored in the extracting part 171. Alternatively, they may be registered and modified in the extracting part 171 by a predetermined operation. And, then the extracting part 171 outputs the extracted monitored results to the disposing part 172.

The axis setting part 173 sets parameters of a vertical axis and a horizontal axis for displaying the monitored results in a chart. The axis setting part 173 preferably represents cyclic time series by one axis and time series within a cycle of the cyclic time series by the other axis. Specifically, it is preferable to represent days (1 to 10 days) by a horizontal axis as one axis and time (0 to 24 hours) by a vertical axis as the other axis. In other examples, it is preferable to represent weeks (first to fourth week) or years (first to ten years) by the horizontal axis and a week (Sunday to Saturday) or month (January to December) by the vertical axis, respectively. The parameters represented by the horizontal axis and vertical axis may be stored in the axis setting part 173 preliminarily. Alternatively, the axis setting part 173 may display an entry screen (not shown) in the display part 148 for entering the parameters of the horizontal axis and the vertical axis (or parameters of at least one of the axes). In this case, the display data producing part 174 preliminarily stores display data for displaying the entry screen and the axis setting part 173 inputs a control signal into the display data producing part 174 for displaying the entry screen. The axis setting part 173 receives an operation signal in accordance with the entry screen from the operating part 140 and sets or modifies the parameters of the horizontal and vertical axes.

The disposing part 172 produces a signal for displaying the monitored results in the chart by using the parameters of the horizontal and vertical axes set by the axis setting part 173 and the monitored results inputted from the extracting part 171. Specifically, the disposing part 172 disposes marks representing the type data stored in the field 283 of the monitored results 280 with respect to the respective monitored results at locations corresponding to the recording time stored in the field 282 in the chart defined by the horizontal and vertical axes according to the above-mentioned parameters. For example, marks “A”, “B”, “C”, “D” and “E” are preliminarily stored as marks representing the type data in relation to “normal sinus rhythm”, “bradycardia”, “tachycardia”, “supraventricular premature beat”, and “premature ventricular contraction”, respectively.

The disposing part 172 outputs a signal representing the processing results to the display data producing part 174. The mark are not limited to “A” to “E”. It is enough to distinguish one mark from another mark. For example, different color marks with the same shape, different sized marks, and marks different motions (animation) may be used. Further, the severity levels are different in the types of the “normal sinus rhythm”, “bradycardia”, “tachycardia”, “supraventricular premature beat”, and “premature ventricular contraction.” Thus, the marks “A”, “B”, “C”, “D” and “E” in the order of the low severity level or marks visually representing the severity level may be assigned. By displaying marks in the order of severity level, the change in patient's status and trends can be visually and easily recognized even when the patient himself or herself, etc. other than a doctor (professional) sees it. Accordingly, the patient may easily recognize cyclic problems and trends and correspondingly respond to them appropriately (take a precaution against them).

The display data producing part 174 produces display data according to the signal inputted from the disposing part 172 and perform processing for displaying the display data in the displaying part 148. The processing here is not limited to a specific one. A standard display processing can be adopted.

### (Display Processing)

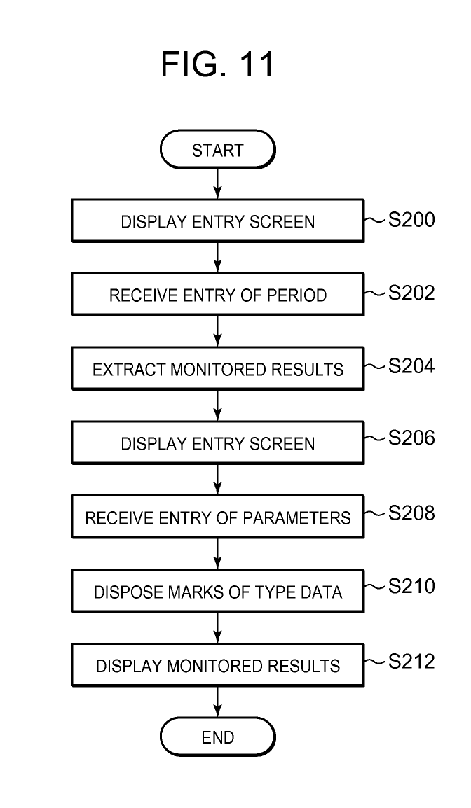

FIG. 11 is a flowchart illustrating a displaying process of monitored results in a portable electrocardiograph 100 according to one embodiment of the invention.

S200: With reference to FIG. 11, the CPU 154 functioning as the extracting part 171 display an entry screen in the displaying part 148 for entering the period of the monitored results to be displayed.

S202: Then, the CPU 154 receives an operating signal representing the period a user set through the entry screen from the operating part 140, and identifies the period according to the operating signal.

S204: Subsequently, the CPU 154 functioning as the extracting part 171 extracts the monitored results within the identified period from the monitored results stored in the nonvolatile memory 155c.

S206: Then, the CPU 154 functioning as the axis setting part 173 display an entry screen for entering parameters of at least one axis in the display part 148.

S208: Then, the CPU 154 receives the parameters of the axis the user inputs through the entry screen and sets the horizontal and vertical axes by using the parameters.

S210: The CPU 154 functioning as the extracting part 171 disposes the marks representing the type information included in the monitored results according to the monitored results extracted in step S204 at the location in the chart defined by the horizontal and vertical axes set in step 208 corresponding to the recording time included in the monitored results, and produces a signal representing the disposing results.

S212: The CPU 154 functioning as the display data producing part 174 produces display data for displaying the monitored results according to the signal produced by the step 210 and displays it in the displaying part 148.

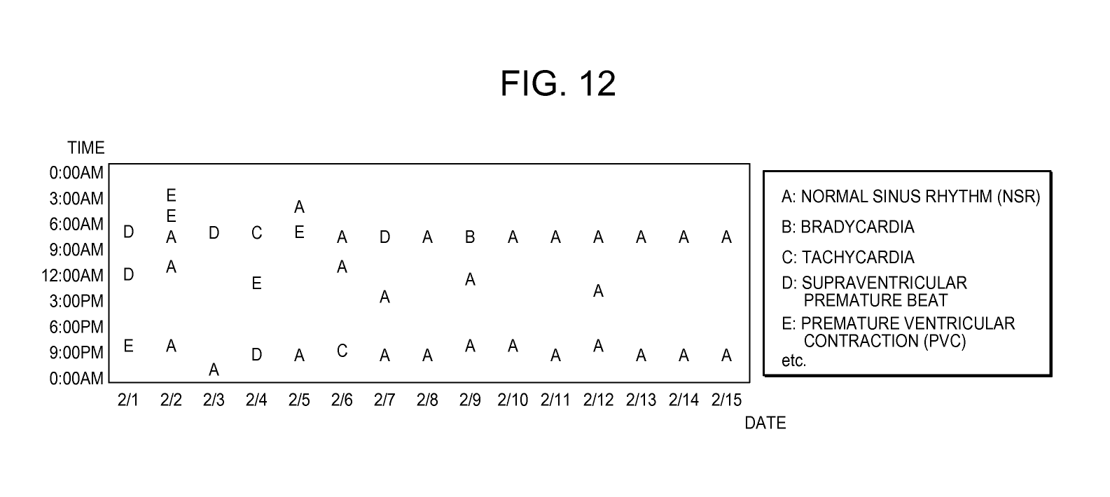

FIG. 12 is a view of a display example of the monitored results screen displayed in a display part 148.

With reference to FIG. 12, the CPU 154 displays a chart 190 plotting the marks “A” to “E”, for example defined by a horizontal axis representing days and a vertical axis representing time. Alternatively, a chart plotting ‘A” to “E” representing severity levels may be displayed instead of the above-mentioned type information.

The above-mentioned processing being performed by the portable electrocardiograph 100 according to the first embodiment, the types of the electrocardiographic waveform monitored are plotted in the chart defined by one axis representing cyclic time series and the other axis representing time series within a cycle of the cyclic time series and displayed efficiently in the portable electrocardiograph 100. Therefore, even when the monitored results by the portable electrocardiograph 100 reaching as many as some hundreds, they are efficiently displayed, thus a medical doctor or the subject himself or herself who reviews the display may visually and easily recognize the status changes and trends of the subject during the corresponding period. In this way, a medical doctor or the subject himself or herself can recognize the relapse after an operation and the effect level of a medication. Further, they can easily recognize a trend of cyclic problems (for example problems occur every day in early mornings). If the person who reviews the display is a doctor or professional giving a medical treatment, he or she can recognize critical hours to which attention should be paid in the medical treatment. Further, more effective medical treatment may be adopted than otherwise. If the person who reviews the display is the subject himself or herself, the subject may adopt a countermeasure to avoid developing to serious diseases corresponding to the trend of his or her own.

### Second Embodiment

### Configuration of Biological Waveform Display System

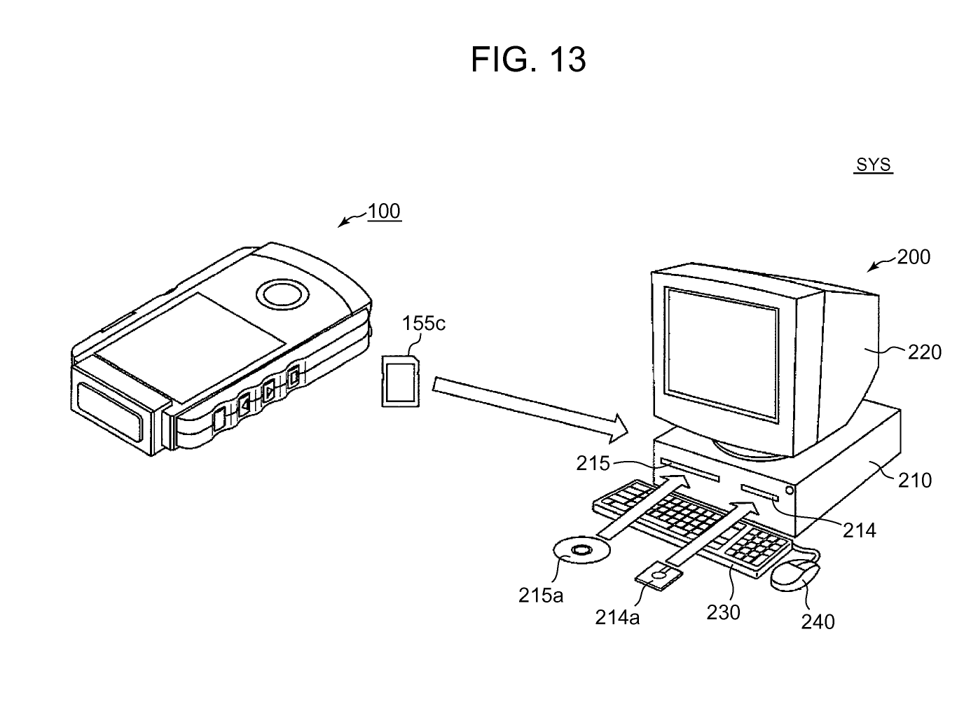

FIG. 13 is a schematic framework of an electrocardiographic waveform display system SYS, a typical example of a biological information display system according to a second embodiment of the invention.

With reference to FIG. 13, the electrocardiographic waveform display system SYS is provided with the portable electrocardiograph 100 and a display unit 200. The portable electrocardiograph 100 monitors electrocardiographic waveforms according to the operation described in the first embodiment. The above-mentioned monitored results are stored, for example in the nonvolatile memory 155c detachable to the portable electrocardiograph 100 while the nonvolatile memory 155c is attached to the display unit 200 to transfer the monitored results of the portable electrocardiograph 100 to the display unit 200.

The transfer of the monitored results from the portable electrocardiograph 100 to the display unit 200 is not limited to the transfer by recording the monitored data in the nonvolatile memory 155c, but other transfer methods may be applied. Specifically, if the portable electrocardiograph 100 is provided with a function of communicating information with other apparatuses through a private line, the monitored results may be transferred from the portable electrocardiograph 100 to the display unit 200 by connecting the portable electrocardiograph 100 to the display unit 200 by using the private line (for example, USB (Universal Serial Bus)). Similarly, the transfer of the monitored results from the portable electrocardiograph 100 to the display unit 200 may be performed by means of a wireless communication such as infrared communication.

The display unit 200 displays a chart in the monitor part 220 according to the monitored results after receiving the monitored results stored in the nonvolatile memory 155c.

The appearance and structure of the portable electrocardiograph 100 is the same as those shown in FIGS. 1 and 2 and it function structure is the same as what is shown in FIG. 5. The monitoring method of the electrocardiographic waveform is the same as those shown in FIGS. 3 and 4. The time setting of the timer included in the CPU 154 (not shown) is preferably not made by the control signal from the CPU 154. The time setting of the timer included in the CPU 154 is preferably made in accordance with the control signal inputted from the display unit 200 through the above-mentioned communication. In the portable electrocardiograph 100, the monitor processing is performed as shown in FIG. 6 and the monitored results is stored in the nonvolatile memory 155c as shown in FIG. 9. Alternatively, the CPU 154 may perform the time setting of its timer to automatically synchronize with the timer of the display unit 200 when the above-mentioned communication between the portable electrocardiograph 100 and the display unit 200 starts through the private line such as the USB cable. In this way, the reliability of the time information with regard to the monitored results stored in the portable electrocardiograph 100 is secured.

In the second embodiment, the CPU 154 functioning as the adding part 154b of the portable electrocardiograph 100 adds the ID data for identifying the portable electrocardiograph 100 itself to the monitored results as the attribute information. The ID data for identifying the portable electrocardiograph 100 itself is preliminarily stored in a predetermined area of the ROM 155a. And it is retrieved from the predetermined area to be added to the monitored results when the CPU 154 functioning as the adding part 154b adds the attribute information to the monitored results. Therefore, in the second embodiment, the ID data for identifying the portable electrocardiograph 100 itself is also stored in the ID data field 281 of the monitored results 280 stored in the nonvolatile memory 155c.

### (Function Structure of Display Unit)

With reference to the FIG. 13, the display unit 200 is typically constituted by a computer further including the computer body 210 having FD (Flexible Disk) drive 214 and CD-ROM (Compact Disk-Read Only Memory) drive 215, keyboard 230 and mouse 240.

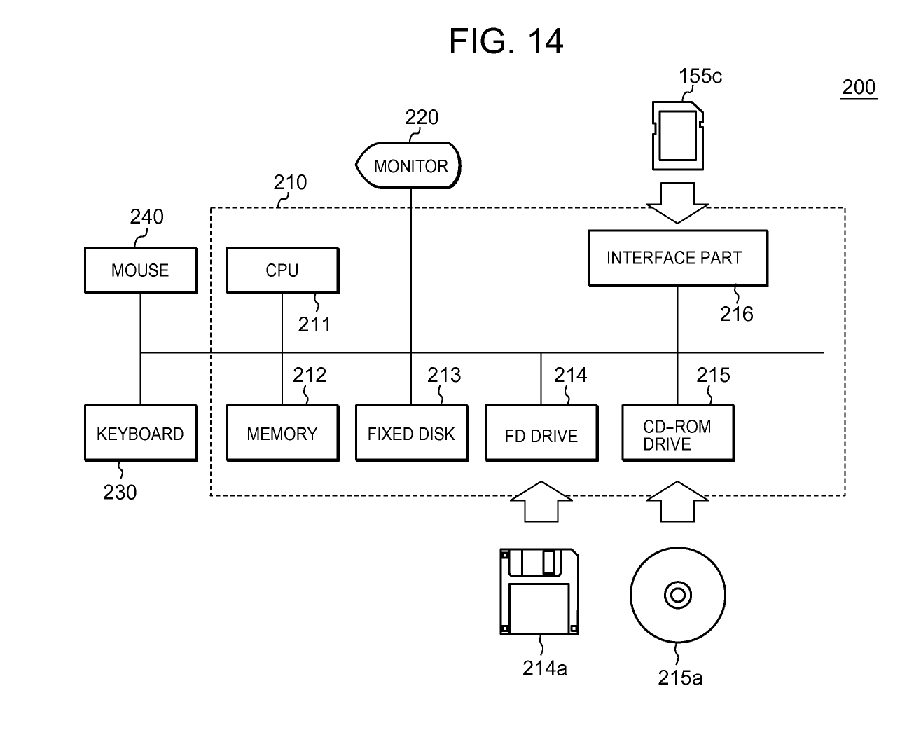

FIG. 14 is a view of schematic configuration illustrating a computer hardware constituting a display unit 200 according to the present embodiment of the invention.

With reference to FIG. 14, the computer body 210 includes the CPU 211, memory 212, fixed disk 213 as a memory and interface part 216 connected to each other through a bus in addition to FD drive 214 and CD-ROM drive 215 as shown in FIG. 13.

The FD 214a is attached to the FD drive 214. The CD-ROM 215a is attached to the CD-ROM drive 215. The display unit 200 is realized by CPU 211 executing a software using the computer hardware such as the memory 212. Generally, such software is circulated stored in a recording medium such as FD 214a or CD-ROM 215a or through a network. And, such software is read by the FD drive 214 or CD-ROM drive 215, etc from the recording medium or received at a communication interface (not shown) to be stored in the fixed disk 213. Further, it is read from the fixed disk 213 to the memory 212 to be executed by the CPU 211.

The monitor part 220 is a display part for displaying information such as biological information CPU 211 outputs, constituted by, for example LCD (Liquid Crystal Display) or CRT (Cathode Ray Tube). The mouse 240 receives an instruction from a user (typically a doctor) corresponding to the operation such as click or slide. The keyboard receives an instruction from a user corresponding to the operation of keys. The CPU 211 is an arithmetic processing part for performing various operations by successively executing programmed commands. The memory 212 stores various data according to the execution of a program by the CPU 211. The interface part 216 is a part for receiving the monitored results with attribute information from the memory 155 of the portable electrocardiograph 100, constituted by the slot to which the nonvolatile memory 155c is attachable and the peripheral circuit controlling the slot, etc in the present embodiment. The communication interface part may be configured to data communicate with the portable electrocardiograph 100 instead of the slot to which the nonvolatile memory 155c is attachable. The fixed disk 213 is a nonvolatile memory for storing the program the CPU 211 executes or the electrocardiographic waveform data with the attribute information received from the memory 155 of the portable electrocardiograph 100. The display unit 200 may be connected to other output devices such as a printer as necessary.

### (Function Structure of Display Unit)

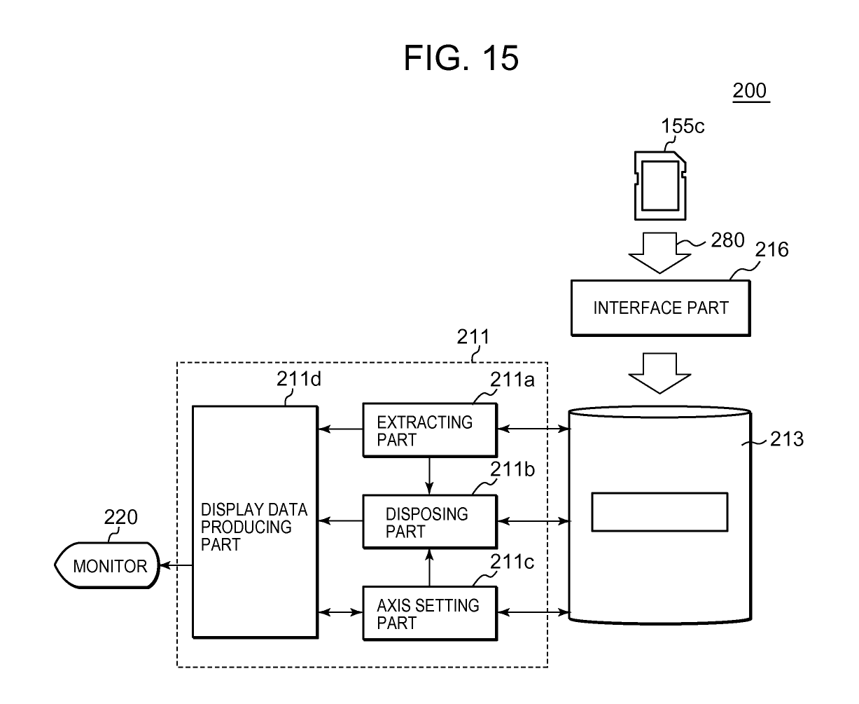

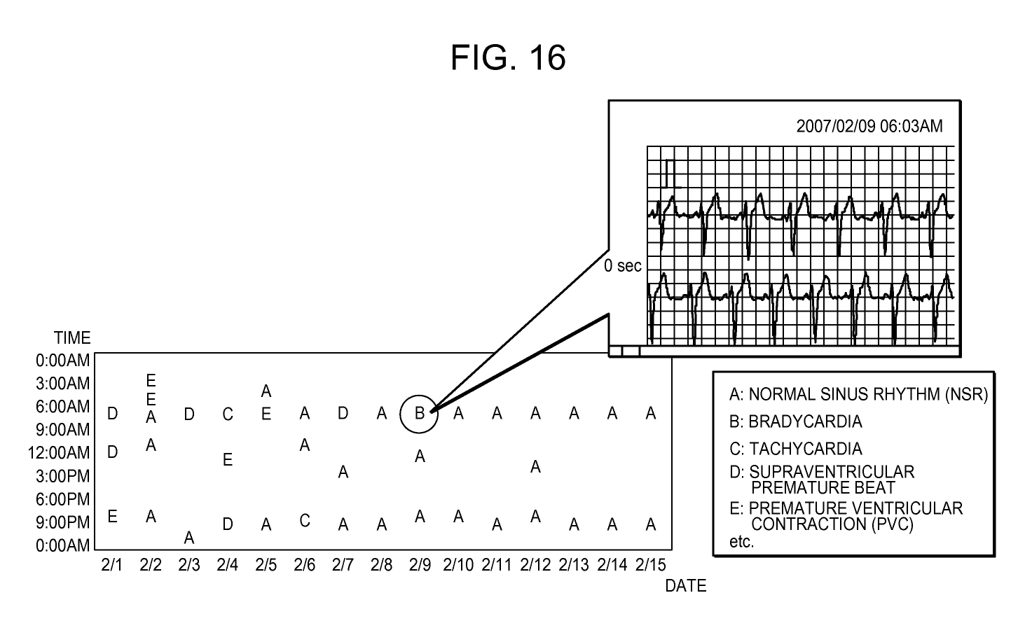

FIG. 15 is a functional block diagram illustrating a functional structure for a display part 200 according to one embodiment of the invention.

With reference to FIG. 15, the CPU 211 of the display unit 200 includes as its functions the extracting part 211a, disposing part 211b, axis setting part 211c and the display data producing part 211d. These functions are the same as those of the extracting part 171, disposing part 172, axis setting part 173 and the display data producing part 174 included in the CPU 154 of the portable electrocardiograph 100 according to the first embodiment respectively described with reference to FIG. 10. The interface part 216, when the nonvolatile memory 155c is attached, reads from the nonvolatile memory 155c the monitored results 280 as shown in FIG. 9 and stores them in the fixed disk 213. At that time, the monitored data is stored in the memory area preferably corresponding to the portable electrocardiograph based on the ID data for identifying the portable electrocardiograph stored in the ID data field 281.

### (Display Processing)

The display unit 200 according to the present embodiment displays in the monitor part 220 the monitored results as shown in FIG. 12 after the same display processing is performed as that of the monitored results in the portable electrocardiograph 100 described with reference to FIG. 11.

The electrocardiographic waveform display system SYS according to the second embodiment is applied, for example when the portable electrocardiograph 100 is brought to display the monitored results with the display unit a doctor uses. The above-mentioned processing is performed by applying the electrocardiographic waveform display system SYS to display efficiently in the display unit 200 by plotting the types of the electrocardiographic waveforms monitored by the portable electrocardiograph 100 in a chart defined by one axis representing cyclic time series and the other axis representing time series within a cycle of the cyclic time series. Therefore, even when the number of monitored results in the portable electrocardiograph 100 reaches some hundreds, a medical doctor reviewing the monitored results through the display unit 200 can visually and easily recognize the status changes and trends of the subject during the corresponding period thanks to the efficient display in the display unit 200. In this way, a doctor or the subject himself or herself can recognize a relapse after an operation or the effect level of the medication. Moreover, the trend of cyclic problems (for example, problems occur every day in the early mornings) can be easily recognized. If the person who reviews the display is a doctor or a professional giving a medical treatment, he or she can recognize the time zone attentions should be paid to in the medical treatment. They can adopt more effective treatment than otherwise. Furthermore, if the person who reviews the display is the subject himself or herself, he or she may adopt a countermeasure to avoid developing serious diseases corresponding to his or her own trends.

### Modified Embodiment

In the display unit 200 according to the second embodiment, the display processing for displaying an electrocardiographic waveform is further performed upon receiving the operation of selecting the mark displayed in the monitored results screen (for example an operation of double-clicking) by the mouse 240 or keyboard 230.

In the modified embodiment, the CPU 211 functioning as the extracting part 211a identifies the monitored results according to the operating signal inputted from the keyboard 230 and outputs the waveform stored in the waveform field 284 of the monitored results to the display data producing part 211d. The CPU 211 functioning as the display data producing part 211d preliminarily stores the horizontal and vertical axes for displaying the waveform to display the waveform inputted from the extracting part 211a in the monitor part 220.

FIG. 16 is a view illustrating a display example of monitored results displayed in the monitor part 220. For example, in the monitored result screen as shown in FIG. 12, when the mark “B” highlighted by circle in FIG. 16 is selected by double-clicking, the above-mentioned processing is performed to display the waveform stored in the waveform data field 284 of the monitored results corresponding to the mark “B” as shown in FIG. 16. In the example shown in FIG. 16, the waveform corresponding to the selected mark is popped up and shown over the chart plotting marks (FIG. 12). However, the mode of displaying the waveform is not limited to the one a shown in FIG. 16. For example, the screen may be changed to display only the waveform.

The above-mentioned modified embodiment describes the display processing performed in the display unit 200 according to the second embodiment. However, in the case that the mark plotted in the chart shown in FIG. 12 is selected in the operation of the portable electrocardiograph 100 according to the first embodiment, the same display processing may be performed in the portable electrocardiograph 100.

The configuration described above makes it possible to review detailed information (waveform data) for necessary monitored results while visually and easily reviewing the monitored results during the predetermined period.

In the above-mentioned embodiment, a portable monitoring device is described as the portable electrocardiograph. A monitoring device for monitoring other biological information such as sphygmomanometer or sphygmograph can produce the same display as the electrocardiograph with the same configuration.

Furthermore, a display program may be provided for a computer to perform a display processing in the above-mentioned portable electrocardiograph 100 or the display unit 200 etc. Such a program may be stored in the computer readable recording medium such as a FD (Flexible Disk), CD-ROM (Compact Disk-Read Only Memory), RAM (Random Access Memory) and a memory card and provided as a program product. Alternatively, such a program may be stored in a recording medium such as a hard disk built in the computer and provided. Further, the program may be provided through downloading from a network.

The program according to the invention may be executed in cooperation with an appropriate program module provided as a part of the operation system (OS) that is retrieved in a predetermined array and at a predetermined timing to perform the processing. In this case, the module is not included in the program itself and the processing is performed in cooperation with the OS. Such a program as not including the module may be included in the program according to the invention.

In addition, the program according to the invention may be incorporated in other programs as a part of the other programs and provided. Also, in this case, the modules included in the other programs are not included in the program itself and the processing is performed in cooperation with the other programs. Such a program as included in other programs may be included in the program according to the invention.

The provide program product is installed in a program storing part such as a hard disk and executed. The program product includes the program itself and the recording medium storing the program.

The embodiments as described above are all examples which should not be taken to limit the scope of the invention. The scope of the invention is to be defined not by the above description but by claims and intended to include all equivalents and modifications without departing from the scope of the invention.

1. An apparatus for monitoring biological information of a subject, comprising:
a detection part configured to detect a signal indicative of the biological information of the subject;
a judging part configured to judge the biological information to identify an attribute of the biological information;
a storage part configured to store the attribute of the biological information together with a time that the attribute is stored; and
a producing part configured to produce a signal to display the attribute in a chart defined by a first axis representing a cyclic time series and a second axis representing a time series within a cycle of the cyclic time series.

2. The apparatus according to claim 1, further comprising a memory part configured to store a threshold used in the judging part, wherein the judging part judges the biological information to identify the attribute of the biological information by comparing the biological information with the threshold.

3. The apparatus according to claim 2, wherein the biological information is an electrocardiographic waveform and the judging part judges the electrocardiographic waveform to identify the attribute of the electrocardiographic waveform as representing a type of arrhythmia.

4. The apparatus according to claim 1, further comprising a selection part configured to select the attribute displayed in the chart, wherein the storage part stores the biological information together with its attribute and the producing part produces a signal to display the biological information upon selection of the attribute in the chart by the selection part.

5. The apparatus according to claim 1, further comprising a setting part configured to set at least one of the first and second axes.

6. A method of displaying biological information obtained by an apparatus for monitoring biological information, comprising the steps of:
obtaining an attribute of the biological information from a storage part together with a time that the attribute is stored; and
producing a signal to display the attribute in a chart defined by a first axis representing a cyclic time series and a second axis representing a time series within a cycle of the cyclic time series.

7. The method according to claim 6, further comprising a step of designating a period of display, wherein the attribute of the biological information stored within the designated period is obtained.

8. The method according to claim 6, further comprising:
a step of detecting a signal indicative of the biological information of a subject;
a step of judging the biological information to identify the attribute of the biological information based on the electric signal, and
a step of storing in the storage part the attribute together with a time that the attribute is stored.

9. The method according to claim 6, wherein the biological information is an electrocardiographic waveform and the attribute represents a type of arrhythmia.

10. A data storage medium configured to record a computer program for displaying biological information obtained by an apparatus for monitoring biological information, the computer program, when executed by a computer, causing the computer to perform a method comprising:
a step of obtaining an attribute of the biological information together with a time that the attribute is stored, from a storage part of the computer; and
a step of producing a signal to display the attribute in a chart defined by a first axis representing a cyclic time series and a second axis representing a time series within a cycle of the cyclic time series.

11. The apparatus according to claim 1, wherein the attribute is displayed in the chart as a symbol representing a type of arrhythmia.

12. The apparatus according to claim 1, wherein the attribute is displayed in the chart as a symbol representing a severity level.

13. The method according to claim 6, wherein the attribute is displayed in the chart as a symbol representing a type of arrhythmia.

14. The method according to claim 6, wherein the attribute is displayed in the chart as a symbol representing a severity level.
